SEQUENCE LISTING (5 sequences)

SEQ ID NO: 1 - DNA, 331 bp, Artificial Sequence
  Description of Artificial Sequence Yeast alph-mating factor prepro leader sequence/Limulus ENP fusion protein.
gaggctgaagctgacggtatctggacccaattgattttcactttggttaacattttggccaccttatggcagtccggtgattttcaattcttggaccacgaatgtcactacagaatcaagccaactttcagaagattgaagtggaaatataagggtaaattttggtgtccatcttggacctctattactggtagagctaccaagtcttctagatccggtgctgtcgaacactctgttagaaacttcgtcggtccagctaagtcttccggtttgatcactgaaagacaagctgaacaattcatttctcaatacaactgataagcttgaattc

SEQ ID NO: 2 - protein, 105 aa, Artificial Sequence
  Description of Artificial Sequence Yeast alpha-mating factor prepro leader sequence/Limulus ENP fusion protein.
EAEADGIWTQLIFTLVNILATLWQSGDRQFLDHECHYRIKPTFRRLKWKYKGKFWCPSWTSITGRATKSSRSGAVEHSVRNFVGPAKSSGLITERQAEQFISQYN

SEQ ID NO: 3 - protein, 27 aa, Limulus polyphemus
ECHYRIKPTFRRLKWKYKGKFWCPSWT

SEQ ID NO: 4 - DNA, 303 bp, Limulus polyphemus
gatggtatttggactcaattgatttttactttggttaataatttggctactttgtggcaatctggtgattttcaatttttggatcatgaatgtcattatagaattaaaccaacttttagaagattgaaatggaaatataaaggtaaattttggtgtccatcttggacttctattactggtagagctactaaatcttctagatctggtgctgttgaacattctgttagaaattttgttggtcaagctaaatcttctggtttgattactcaaagacaagctgaacaatttatttctcaatataat

SEQ ID NO: 5 - protein, 101 aa, Limulus polyphemus
DGIWTQLIFTLVNNLATLWQSGDFQFLDHECHYRIKPTFRRLKWKYKGKFWCPSWTSITGRATKSSRSGAVEHSVRNFVGQAKSSGLITQRQAEQFISQYN

(19) United States
(12) Patent Application Publication
(10) Pub. No.: US 2003/0087823 A1
(43) Pub. Date: May 8, 2003

(54) Compositions comprising endotoxin neutralizing protein and derivatives and uses thereof

(75) Inventors: Erik J. Paus, Brewster, MA (US); Norman R. Wainwright, Falmouth, MA (US)

Correspondence Address: PENNIE & EDMONDS LLP, 1155 Avenue of Americas, New York, NY, 10036-2711, US

(73) Assignee: Associates of Cape Cod, Inc.

(21) Appl. No.: 10/241,173

(22) Filed: Sep. 11, 2002

Related U.S. Application Data
(63) Continuation of application No. 09/109,175, filed on Jul. 2, 1998.

(51) Int. Cl. A61K 38/17; A61K 31/545; A61K 31/407

(52) U.S. Cl. 514/12; 514/200; 514/192; 514/210.09

(57) ABSTRACT

The present invention relates to the use of endotoxin neutralizing protein (ENP) and derivatives thereof as stand alone microbial inhibitors or as synergistic enhancers of antibiotics and preservatives. Compositions comprising ENP or alone or in combination with an antibiotic may be used to prevent or treat gram-negative bacterial infections, endotoxemia, septic shock, gram-positive bacterial infections, yeast infections and fungal infections.

This is a continuation of application Ser. No. 09/109,175 filed Jul. 2, 1998.

### 1. FIELD OF THE INVENTION

The present invention generally relates to endotoxin neutralizing proteins (ENPs) of horseshoe crabs. The invention more specifically relates to therapeutic methods for preventing or treating microbial infections, by administering endotoxin neutralizing protein (ENP) or ENP-derivatives alone, or in combination with antibiotics. The invention also relates to pharmaceutical compositions comprising ENP or ENP-derivatives that can be used in connection with such methods.

### 2. BACKGROUND OF THE INVENTION

2.1. Endotoxins

Endotoxins are high molecular weight lipopolysaccharide (LPS) complexes consisting lipid, carbohydrate, and protein, and are characterized by an overall negative charge and heat stability. Endotoxins are constituents of the outer cell wall of gram-negative bacteria. LPS has three distinct chemical domains: an innermost phospholipid (lipid A) moiety, an intermediate core polysaccharide, and the outermost O-specific polysaccharide side chain.

Endotoxins are released when gram-negative bacteria are disrupted, such as that during antibiotic therapy, or when bacteria grow or are lysed within a host organism. In mammals, endotoxins are potent inducers of inflammatory responses. LPS of endotoxins induces release of mediators, such as cytokines, from host inflammatory cells. Systemic exposure to endotoxins causes uncontrolled, deleterious inflammatory responses such as those associated with septic shock, endotoxemia and may ultimately result in intravascular coagulation, and failure of various vital organs.

2.2. Endotoxin Neutralizing Protein

Endotoxin neutralizing protein (ENP; also known as anti-LPS factor) are found in cellular lysate of horseshoe crab amebocytes. ENP binds and neutralizes endotoxins. This is, ENP binding significantly reduces or abolishes endotoxins' ability to induce inflammatory responses in animals. ENPs are present in amebocytes of all four horseshoe crab species: Limulus polyphemus, Tachypleus gigas, Tachypleus tridentatus, and Carcinoscropius rotundicauda.

The genetics of ENP remains obscure. ENP may be encoded by a small gene family with members that exhibit microheterogeneity at the nucleotide and amino acid levels. ENPs from the various horseshoe crab species are evolutionarily related and exhibit approximately 70% homology at the amino acid sequence level.

ENP plays a key role in the anti-infection pathway of horseshoe crabs. When exposed to endotoxins from gram-negative bacteria, ENP present in the hemolymph binds the LPS of the infecting bacteria to form clots that trap the invading bacteria, preventing intravascular coagulation.

The neutralizing activity of ENP is due to its high affinity binding to the lipid A moiety of endotoxins. ENP is an amphipathic single chain protein with clustering of hydrophobic amino acids at the amino terminal and an array of basic amino acids in the central disulfide-bonded loop region. The exact mode of ENP's binding of endotoxins remains unclear. It is likely, however, that the hydrophobic and cationic amino acids of ENP interact respectively with the fatty acid chains and the phosphate groups of the lipid A moiety.

International publication WO 92/20715 discloses that ENP has a core domain that is essential to endotoxin binding and neutralization. In Limulus polyphemus ENP (also known as Limulus anti-LPS factor or Limulus ENP), this domain comprises a hydrophobic loop from amino acid residues 30 to 56 (Kloczewiak et al., 1994, J. Infectious Diseases 170:1490-1497).

Studies have shown that ENP binds to and inactivates endotoxins from a wide range of gram-negative bacteria, such as Klebsiella pneumonias, Serratia marcescens, Salmonella enteritidis, Escherichia coli 0113 wild type, Escherichia coli rough mutant (J-5), Salmonella abortus equi, and lipid A from Salmonella minnesota Re 595 (WO 92/20715). In these studies, ENP was mixed with endotoxins or lipid A at various ratios and the endotoxin activity in the mixture measured. In all cases where the ENP was added in excess, endotoxin activity was greatly reduced.

ENP has been shown to inhibit endotoxin induction of various cellular processes and events such as mitogenesis of murine splenocytes (Warren, 1992, Infect. Immun. 60:2506-2513), activation of human endothelial cells (Desch et al., 1992, Infect Immun. 57:1612-1614) and release of tumor necrosis factor by human macrophages (Kuppermann et al., 1994, J. Infect. Dis. 170:630-635).

ENP also inhibits the growth of rough gram-negative bacteria (Morita et al., 1985, J. Biochem. 97: 1611-1620). ENP has potent endotoxin neutralizing activity in vivo. ENP is protective against lethal Escherichia coli endotoxin challenge in rats (Wainwright et al., Cellular and Molecular Aspects of Endotoxin Reactions, Nowotny et al. eds., Amsterdam: Elsevier Science, pp. 315-325 (1990)), and against lethal meningococcal challenge in rabbits (Alpert et al., 1992, J. Infect. Dis. 165:4.94-500). ENP treatments also improve the survival rate of rabbits and rats with E. coli sepsis (Saladino et al., 1994, Circ. Shock 42:104-110; Kuppermann et al., 1992, Pediatr. Res. 31:32A).

ENP exhibits very potent endotoxin neutralizing activity in vivo. This has been demonstrated in a rat model system, wherein the rats were infected with an encapsulated strain of E. coli that is virulent in humans. ENP at a dose of 50 mg/kg blocked the lethal effect of endotoxins released by the infecting E. coli even when ENP was administered after endotoxin had circulated in the animals and was likely to have bound to target cells. When compared with animals treated with anti-endotoxin antibody HA-1A, animals treated with ENP showed significantly lower circulating endotoxin concentrations and improved survival rates (Kuppermann et al., 1994, J. Infect. Dis. 170:630-635).

Similar protective effects by ENP also have been demonstrated in rabbits. ENP administered at a dose of either 2.5 or 5 mg/kg before lethal endotoxin challenges significantly improved physiological functions and survival rate. ENP treatment also attenuated the toxic effect of E. coli endotoxins and improved survival rates, even when administered 30 min. after endotoxin challenge. In that instance, the protective effect of ENP was weaker than that seen in animals pretreated with ENP before endotoxin challenge (Gacia et al., 1994, Care Med. 22:1211-1218).

Citation or identification of any reference herein shall not be construed as an admission that such reference is available as prior art to the present invention.

### 3. SUMMARY OF THE INVENTION

The present invention generally relates to uses of and compositions comprising endotoxin neutralizing protein (ENP) of horseshoe crabs and ENP-derivatives. The invention provides novel therapeutic methods for preventing or treating microbial infections comprising administering an effective amount of one or several ENPs or ENP-derivatives alone, or in combination with an effective amount of one or several antibiotics. The invention also provides pharmaceutical compositions comprising one or more ENPs or ENP-derivatives alone, or combined with one or more antibiotics, which compositions may be used in the methods of the invention. The methods and compositions of the invention may be used to prevent or treat infections caused by gram-negative, bacterial spp., gram-positive bacteria spp., yeast spp. and fungal spp.

The invention also relates to recombinant production of ENP and ENP-derivatives. The invention provides methods for expressing ENP and ENP-derivatives in Saccharomyces and Pichia cells and isolating ENP and ENP-derivatives therefrom.

3.1. Definitions

In order to provide a clear and consistent understanding of the specification and claims, including the scope to be given to a term, the following definitions are given to various terms and abbreviations used herein.

“Antibiotic” means a drug produced by microorganisms and/or synthetically that at low concentrations can inhibit the growth or kill a microbial organism, such as bacteria, yeast and fungi.

“Effective amount” means an amount of an agent sufficient to kill a targeted microbe, to control or prevent the spread of infection by the microbe, or to prevent, inhibit or retard the growth of the microbe. When a method comprises administering several agents, an effective amount of a particular agent refers to the amount of that agent, in combination with the other agent(s), sufficient to kill the microbe, to control or prevent the spread of infection by the microbe, or to prevent, inhibit or retard the growth of the microbe.

“Endotoxins” means pyrogenic toxins comprising LPS of a gram-negative bacterium.

“Endotoxin inactivating protein or EIP” means a protein or peptide that binds and neutralizes endotoxins. EIP may be a native endotoxin neutralizing protein (ENP), such as Limulus ENP, found in amebocyte lysates of any species of horseshoe crab. EIP may also be derived from a native ENP. Such ENP-derivatives include proteins or peptides comprising (a) a native ENP core sequence that binds and neutralizes endotoxins (e.g., the sequence from residues 30 to 50 of Limulus ENP), or (b) the entire native ENP sequence. ENP-derivatives may be fusion or chimeric proteins comprising amino acid sequences of two or more proteins or peptides.

“Endotoxin neutralizing protein or ENP,” also known as anti-LPS factor and endotoxin binding/neutralizing protein, is a protein found in horseshoe crab amebocyte lysates that binds and neutralizes the biological activity of endotoxins. A species of ENP is the Limulus anti-LPS factor or LALF or Limulus ENP (LENP).

“Fungi” means non-filamentous fungi or filamentous fungi.

“Inhibit” means suppress, arrest, prevent, reduce or retard.

“LB” means Luria broth

“Limulus ENP or anti-LPS factor” means the 11.8 kDa ENP from the horseshoe crab Limulus polyphemus.

“LPS” means lipopolysaccharide from gram-negative bacteria and is a constituent of endotoxins.

“Microbe” means any species of bacteria, yeast or fungi.

“PMB” means polymixin B.

“Patient” means a warmed-blooded animal that is susceptible to or has a microbial infection.

“Sp.” means a single species.

“Spp.” means a plurality of species.

### 4. BRIEF DESCRIPTION OF THE FIGURES

FIG. 1. ENP synergy with polymixin B in inhibiting gram-negative bacterium E. coli. The figure shows the inhibitory effect of increasing concentrations of polymyxin B alone (solid bar) or with 10 μg/ml of Limulus ENP (hatched bar) on the growth of E. coli 25303 in Luria broth.

FIG. 2. ENP synergy with gentamycin sulfate in inhibiting gram-negative bacterium E. coli. The figure shows the inhibitory effect of increasing concentrations of gentamycin sulfate alone (solid bar) or with 20 μg/ml of Limulus ENP (hatched bar) on the growth of E. coli 25303 in Luria broth.

FIG. 3. ENP synergy with gentamycin-sulfate in inhibiting gram-negative bacterium Bortadella sp: The figure shows the inhibitory effect of increasing concentrations of gentamycin sulfate alone (solid bar) or with 20 μg/ml of Limulus ENP (hatched bar) on the growth of Bortadella sp. in FBS.

FIG. 4. ENP synergy with tetracycline in inhibiting gram-negative bacterium E. coli. The figure shows the inhibitory effect of increasing concentrations of tetracycline alone (solid bar) or with 10 μg/ml of Limulus ENP (hatched bar) on the growth of E. coli 25303 in Luria broth.

FIG. 5. ENP synergy with polymixin B in inhibiting gram-positive bacterium S. aureus. The figure shows the inhibitory effect of increasing concentrations of polymyxin B alone (solid bar), or with 10 μg/ml of Limulus ENP (hatched bar) on the growth of S. aureus in Luria broth.

FIG. 6. Map of Saccharomyces cerevisiae expression vector pCGS965 comprising an expression construct encoding a yeast alpha-mating factor prepro leader sequence/Limulus ENP fusion protein.

FIG. 7. Map of Pichia pastoria integration vector pPICZ comprising an expression construct encoding a yeast alpha-mating factor prepro leader sequence/Limulus ENP fusion protein.

FIG. 8. Map of Pichia pastoria integration vector pHIL-D2 comprising an expression construct encoding a yeast alpha-mating factor prepro leader sequence/Limulus ENP fusion protein.

FIG. 9. Map of Pichia pastoria integration vector pAO815 comprising an expression construct encoding a yeast alpha-mating factor prepro leader sequence/Limulus ENP fusion protein.

FIG. 10. Map of Pichia pastoria integration vector pAOGAP comprising an expression construct encoding a yeast alpha-mating factor prepro leader sequence/Limulus ENP fusion protein.

FIG. 11. The nucleotide sequence (SEQ ID NO:1) and amino acid sequence (SEQ ID NO:2) of a yeast alpha-mating factor prepro leader sequence/Limulus ENP fusion protein. The prepro leader sequence is from amino acid residues 1 to 4. The Limulus ENP is from amino acid residues 5 to 105.

FIG. 12. Amino acid sequence of the endotoxin binding and neutralizing domain of Limulus ENP (SEQ ID NO:3).

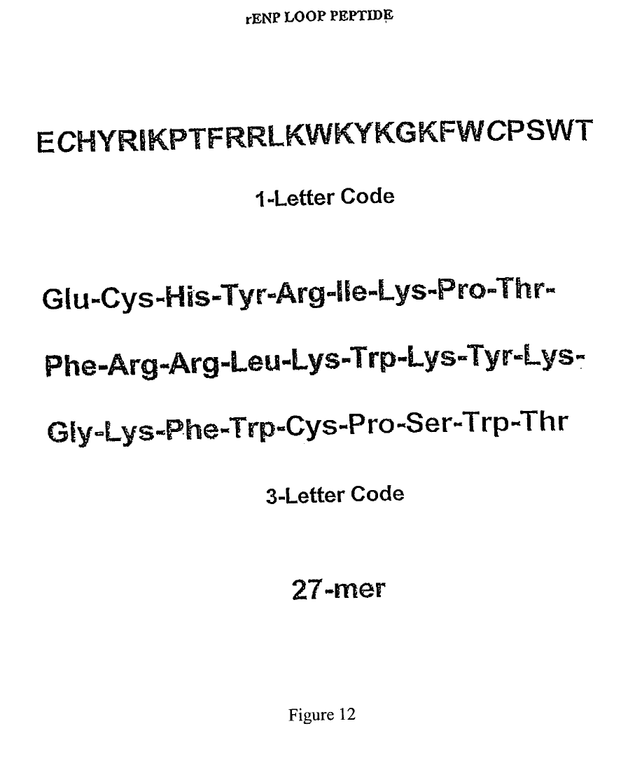

FIG. 13. The nucleotide sequence (SEQ ID NO:4) and amino acid sequence (SEQ ID NO:5) of a Limulus ENP. The amino acid residues 14, 81 and 90 of this Limulus ENP differs from the corresponding residues of the Limulus ENP shown in FIG. 11.

### 5. DETAILED DESCRIPTION OF THE INVENTION

The present invention relates to novel use of ENP and ENP-derivatives as antimicrobial agents as well as synergistic enhancers or potentiators of antibiotics. One aspect of the invention is directed to the use of ENP and ENP-derivatives in preparing novel and more potent pharmaceutical compositions for preventing or treating infections caused by microbes such as bacteria, yeast and fungi. The invention provides novel pharmaceutical compositions that combine one or more ENPs or ENP-derivatives with one or more antibiotics that inhibit gram-negative bacteria. The invention also provides novel pharmaceutical compositions comprising one or more ENPs or ENP-derivatives in combination with one or more antibiotics that inhibit gram-positive bacteria. The invention further provides novel pharmaceutical compositions comprising one or more ENPs or ENP-derivatives in combination with one or more antibiotics that inhibit yeasts or fungi. The invention additionally provides therapeutic methods for preventing or treating microbial infections comprising administering an effective amount of one or more ENPs or ENP-derivatives alone, or in combination with an effective amount of one or more antibiotics that inhibit bacteria, yeast and/or fungi.

A further aspect of the invention is directed to producing ENP and ENP-derivatives in heterologous systems, such as Saccharomyces spp. and Pichia spp. The invention provides methods for constitutive or inducible expression of ENP and ENP-derivatives in Saccharomyces and Pichia in form of fusion proteins comprising a yeast alpha mating factor prepro leader sequence linked to the ENP or ENP-derivative sequence. In the methods of the invention, the fusion proteins are processed and secreted by the host cell to produce ENP or ENP-derivatives. The invention also provides procedures for purifying recombinantly produced ENP and ENP-derivatives as well as yeast transformation and integration vectors containing constitutive or inducible promoters operably linked to sequences encoding fusion protein comprising ENP or ENP-derivatives.

The present invention is based on several surprising discoveries. One unexpected discovery is that ENP acts synergistically with antibiotics in inhibiting gram-negative bacteria. That is, the inhibitory potency of concurrent administration of ENP and an antibiotic is several orders of magnitude greater than that of separate administration of the same amount of either agent. This effect lowers the minimum inhibitory concentration of antibiotics required for effective dosage, and provides for novel and more potent antimicrobial compositions. Such compositions may be used to effectively treat gram-negative bacterial infections employing only low antibiotic dosages. The novel antimicrobial compositions may also be more effective against certain antibiotic resistant bacterial strains. The lower antibiotic dose regimes available with such compositions also may beneficially curtail or slow the development of antibiotic resistant strains amongst clinically significant bacterial pathogens.

Another surprising discovery is that the inhibition of gram-negative bacteria by ENP plus antibiotic compositions produces several orders of magnitude less endotoxins than that detected in inhibition by antibiotics alone. Inhibition of gram-negative bacteria by certain antimicrobial agents, including antibiotics, often is associated with an undesirable side-effect of endotoxin release from the inhibited or killed bacteria. The ability of ENP to significantly reduce such releases greatly enhances the utility of ENP plus antibiotic compositions and methods that use both ENP and antibiotics for treating gram-negative bacterial infections or inhibiting gram-negative bacterial growth as compared to that of conventional compositions comprising antibiotics or antimicrobial agents without ENP. Accordingly, the therapeutic methods and pharmaceutical compositions of the invention have particular advantages for preventing or treating diseases or conditions involving endotoxins such as gram-negative bacteria-induced sepsis or shock.

A further surprising discovery is that compositions comprising ENP have inhibitory activity against gram-positive bacteria, yeast and fungi. This is surprising because heretofore ENP was known to interact only with the LPS of gram-negative bacteria and these other microorganisms have no such constituent. Accordingly, ENP was not expected to interact with gram-positive bacteria, yeast or fungi, much less inhibit any of them. Without intending to limit the present invention to any particular mechanism, it is believed that ENP binding alters the cell membrane permeability of gram-positive bacteria spp., yeast spp. and fungal spp. and that that property accounts for ENP's inhibitory activity against these microorganisms.

In sum, the therapeutic methods and pharmaceutical compositions of the invention have several advantages over conventional antibiotic therapies and compositions. One such advantage is that the methods of the invention require no or less antibiotics to achieve the same level of therapeutic efficacy. This provides the benefit of reducing the reliance on conventional antibiotics for antimicrobial therapy and thereby reducing the opportunity for development of antibiotic-resistant strains. Another advantage is that the methods of the invention reduce the level of free endotoxins that is produced by inhibited or killed gram-negative bacteria. This has the beneficial effect of preventing or reducing the severity of the pyrogenic responses that often occur during suppression of gram-negative bacterial infections.

5.1. Endotoxin Inactivating Proteins

The present invention contemplates the use of one or more ENPs or ENP-derivatives alone, or in combination with one or more antibiotics to prevent or treat infections caused by a variety of microorganisms, including but not limited to gram-negative bacterial spp., gram-positive bacterial spp., yeast spp. and fungal spp. The invention also contemplates the use of ENP or ENP-derivatives alone, or in combination with conventional preservatives as preservatives of cosmetic and personal care preparations.

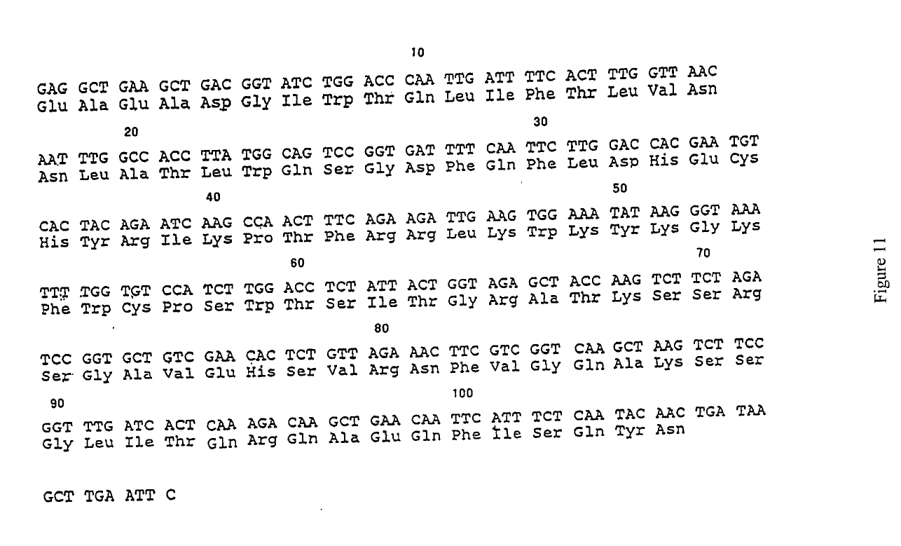

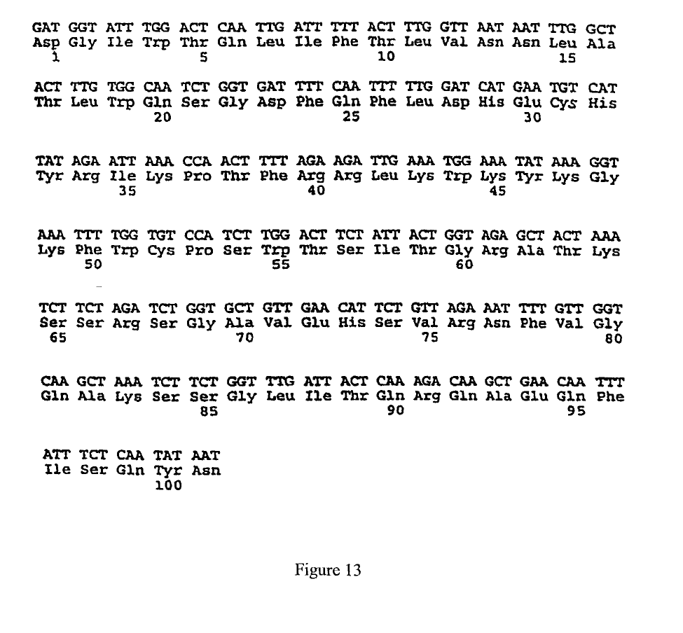

ENPs and ENP-derivatives that may be used for the purpose of the present invention are collectively referred to herein as endotoxin inactivating proteins (ElPs). According to the present invention, an EIP may be a native ENP isolated from the amebocyte lysate of a horseshoe crab. In particular embodiments, useful ENP may be those from any horseshoe crab species, such as Limulus polyphemus, Tachypleus gigas, Tachypleus tridentatus, and Carcinoscropius rotundicauda. In preferred embodiments, ENPs are ENPs from Limulus polyphemus (Limulus ENPS), which are also known as LALF. Limulus ENPs have a molecular weight of about 11.8 kDa (Wainwright et al., Cellular and Molecular Aspects of Endotoxin Reactions, Nowotny et al. eds., Amsterdam: Elsevier Science, pp. 315-325 (1990)). While Limulus ENPs are highly homologous and have a conserved endotoxin binding and neutralizing domain (i.e., the sequence of SEQ ID NO:3), they may exhibit some amine acid sequence heterogeneity outside that domain. The amino acid sequence of a Limulus ENP is that of residues 5 to 105 of SEQ ID NO:2 (shown in FIG. 11), or the sequence of SEQ ID NO:5 (shown in FIG. 13).

ENP may be isolated from horseshoe crabs and purified using methods well known in the art. See U.S. Pat. Nos. 5,614,369, 5,627,266, 5,594,113, 5,747,113; WO 92/20715; WO 89/12644; and Kloczewiak et al., 1994, J. Infect. Diseases. 170:1490-1497. In preferred embodiments, ENP or ENP-derivatives produced from recombinant expression systems are used to practice the present invention.

ENP or ENP-derivatives may be expressed and isolated from any well known recombinant expression systems including but not limited to yeast expression systems, insect cell expression systems, animal and plant cell expression systems, as well as transgenic plant and animal expression systems. In preferred embodiments, yeast expression systems, such as the S. cerevisiae system presented by Kuppermann et al., 1994, J. Infect. Dis., Vol. 170, pp. 630-635, or the Pichia expression system disclosed in Example 1 below, are used to produce ENP or ENP-derivatives.

According to the invention, an EIP also may be certain derivatives of ENP. Useful ENP-derivatives include proteins and peptides comprising the endotoxin binding and neutralizing (EBN) domain of an ENP. Procedures for determining such domain and detecting peptides or proteins having the domain are well known. See, for example, WO 92/20715 and Kloczewiak et al., 1994, J. Infect. Diseases. 170:1490-1497. In preferred embodiments, ENP-derivatives comprise the EBN domain of LALF (i.e., SEQ ID NO:3). ENP-derivatives that comprise an EBN domain may be prepared by proteolytic digests of ENP, followed by isolation and purification of the protein fragments that contain the EBN domain. Alternatively, such ENP-derivatives also may be chemically synthesized based on known amino acid sequence of an ENP and the location of EBN domain therein. Preferably, the ENP-derivatives are recombinantly produced using the expression systems discussed above and those exemplified below. Chemical synthesis and recombinant expression further may be used to produce useful ENP-derivatives that additionally comprise non-ENP sequences such as signal sequences, processing sequences as well as antimicrobial sequences.

According to the invention, ENP or ENP-derivative of any degree of purity may be used to practice the disclosed methods and compositions. In a preferred embodiement, the ENP or ENP-derivative is at least 50% pure by weight. In a more preferred embodiement, the ENP or ENP-derivative is at least 80% pure by weight. In a most preferred embodiment, the ENP or ENP-derivative is at least 95% pure by weight.

5.2. Antibiotics Effective Against Gram-Negative Bacteria

According to the invention, ENP and ENP-derivatives may be beneficially used in combination with antibiotics that inhibit gram-negative bacteria spp. to achieve synergistically enhanced killing or suppression of such bacteria or to drastically reduce endotoxin release resulting from such killing or suppression. Gram-negative effective antibiotics that may be used in the methods and compositions of the invention include, but are not limited to, polymyxin B, ampicillin, amoxicillin, penicillin G, tetracycline, erythromycin, spectinomycin, cefoxitin, trimethoprimsulfamethoxazole, chloramphenicol, rifampin, minocycline, sulfonamide, nitrofurantoin, gentamicin, cefamandole, carbenicillin, ticarcillin, tobramycin, amikacin, cephalosporin, cefoxitin, streptomycin, and clindamycin. Further, the present invention encompasses using more than one gram-negative antibiotic in combination with an ENP or derivative thereof.

The synergy between ENP or ENP-derivatives and gram-negative effective antibiotics means that new antibiotic compositions and therapies can be prepared or developed using lower levels of each type of drug than would be necessary if either type of drug were administered alone. As an example, polymixin B (PMB) is a cyclic, basic polypeptide that binds to and disrupts the lipid A component of endotoxins. PMB inhibits certain biological activities of endotoxins and is a potent drug for treating gram-negative bacterial infections in warm-blooded animals. It has been shown, for example that pretreatment with PMB was effective in preventing shock and mortality in rabbits challenged with a potent dose of E. coli endotoxins (Baldwin et al., 1991, J. Infect. Dis. 164:542-549). However, because of its systemic toxicity, PMB has limited use in treating bacterial infections, except as a topical agent (WO 92/03535 and WO 94/25476). According to the present invention, compositions comprising ENP and PMB can be formulated using lower levels of PMB and still achieve desired therapeutic efficacy due to synergism produced by the inclusion of ENP.

In a further example, ENP or ENP-derivatives may be used in combination with PMB to treat septic shock caused by meningococcemia. In a rabbit model of meningococcemia, pretreatment with PMB alone failed to improve physiologic functions or mortality resulting from challenges with meningococcal endotoxins (Baldwin et al., 1991, J. Infect. Dis. 164:542-549). By contrast, ENP treatment significantly improved various physiological functions and survival rate even when administered 30 minutes after meningococcal endotoxin challenges (Alpert et al., 1992, J. Infect. Dis. 165:494-500).

5.3. Treatment or Prevention of Gram-Negative Bacterial Infections

Therapeutic administration of ENP or ENP-derivatives alone, or in combination with gram-negative antibiotics may be achieved using methods known to those skilled in the art including topical, intravenous, intramuscular or subcutaneous routes, direct delivery into an infected body cavity by infusion, and oral or rectal administration. A therapeutic dose of the ENP or ENP-derivative plus antibiotic composition is an amount that is effective (a) to at least control or inhibit the spread of the bacterial infection, or (b) to prevent or reduce LPS-mediated stimulation of neutrophils and mononuclear cells caused by the infection (e.g., the pyrogenic response).

In a preferred embodiment, the effective amount of ENP or derivative is a concentration of between approximately 0.1 and 100 mg ENP or ENP-derivative per kg of body weight of a patient. As used herein, a patient is a warm-blooded animal, including domestic and farm animals and humans. Other useful ranges include between 0.1 and 1 mg; 1 and 10 mg; and 10 and 100 mg of ENP or ENP-derivative per kg body weight. A typical amount of ENP or ENP-derivative is between approximately 10 and 50 mg of ENP or ENP-derivative per kg body weight.

The dose of gram-negative antibiotics used in combination with ENP or ENP-derivatives may be adjusted up or down based upon known therapeutic doses and routine experimentation by those skilled in the art. The therapeutic effective amounts of the ENP or ENP-derivative plus antibiotic compositions of the invention also may be determined by routine experimentation using known methods and considering the effective dosages discussed above.

5.4. Treatment or Prevention of Gram-Positive Bacterial Infections

The invention also provides for using ENP or ENP-derivatives alone to prevent or treat gram-positive bacterial infections. A therapeutic dose of the ENP or ENP-derivative is an amount that is effective to kill the gram-positive bacteria, or to control or inhibit the spread of the infection. An effective dose can be determined by one skilled in the art using routine experimentation.

In a preferred embodiment, the effective amount of ENP or derivative is a concentration of between approximately 0.1 and 100 mg ENP or ENP-derivative per kg of body weight of a patient. Other useful ranges include between 0.1 and 1 mg; 1 and 10 mg; and 10 and 100 mg of ENP or ENP-derivative per kg body weight. A typical amount of ENP or ENP-derivative is between approximately 10 and 50 mg of ENP or ENP-derivative per kg body weight.

The invention also provides for using ENP or ENP-derivatives in combination with antibiotics that inhibit gram-positive bacteria to prevent or treat gram-positive bacterial infections. Gram-positive effective antibiotics that may be used in the methods and compositions of the invention include, but are not limited to, erythromycin, cephalosporin, chloramphenicol, rifampin, aminoglycosides, vancomycin, amoxicillin, ampicillin, penicillin G, penicillin V, cloxacillin, dicloxacillin, methicillin, nafcillin, oxacillin, piperacillin, meziocillin, cephalexin, cephradine, cefaclor, cefadroxil, cefixime, cefuroxime, cefprozil, cephalothin, cefrazolin, cephapirin, cefoperazone, ceftrixone, gentamicin, tetracycline, chlorotetracycline, oxytetracyclione, demeclocycline, minocycline, methacycline, clindamycin, lincomycin, clarithromycin, azithromycin, metronidazole, metronidazole, and polymyxin B. Further, one or several antibiotics that inhibit gram-positive bacteria may be used with ENP or ENP-derivatives in the therapeutic methods and compositions of the invention.

A therapeutic dose of the ENP or ENP-derivative plus antibiotic composition of the invention is an amount that is effective to kill the gram-positive bacteria, or to control or inhibit the spread of the gram-positive bacterial infection. The concentration of gram-positive antibiotic used in combination with ENP or ENP-derivatives may be adjusted up or down based upon known therapeutic doses and routine experimentation by those skilled in the art. The effective amounts of ENP or ENP-derivatives used in combination with the antibiotics may be those indicated above for using ENP or ENP-derivatives alone to prevent or treat gram-positive bacterial infections.

5.5. Treatment or Prevention of Yeast or Fungal Infections

The invention also provides for using ENP or ENP-derivatives alone, or in combination with antibiotics that inhibit yeasts and fungi to prevent or treat yeast or fungal infections, such as those by Candida parapilosis and C. albicans. Useful antibiotics that may be combined with ENP or ENP-derivatives for such purpose include, but are not limited to, amphotericin B, clotrimazole, flucytosine, griseofulvin, haloprogin, hydroxyslilbamidine, miconazole, nystatin, and tolnaftate. More than one such antibiotic may be used together with one or more ENPs or ENP-derivatives in treating or preventing yeast or fungal infection.

According to the invention, the therapeutic dose of the ENP or ENP-derivative composition or the ENP or ENP-derivative plus antibiotic composition is the amount that is effective to kill the yeast or fungal pathogen, or to control or inhibit the spreading of the yeast or fungal infection. In a preferred embodiment, an effective amount of ENP or ENP-derivative is a concentration of between approximately 0.1 and 100 mg ENP or ENP-derivative per kg of body weight. Other useful ranges include between 0.1 and 1 mg; 1 and 10 mg; and 10 and 100 mg of ENP or ENP-derivative per kg body weight. A typical amount of ENP or ENP-derivative is between approximately 10 and 50 mg of ENP or ENP-derivative per kg body weight.

The concentration of antibiotics used in combination with ENP or ENP-derivative may be adjusted based upon known therapeutic doses and routine experimentation by those skilled in the art. The effective amounts of the ENP or ENP-derivative plus antibiotic compositions of the invention may be determined by routine experimentation using known methods and considering the effective dosages discussed above.

5.6. Therapeutic Methods and Pharmaceutical Compositions

The present invention is directed to methods for preventing or treating microbial infections. The patient or subject treated by the methods of the invention is a warm-blooded animal, preferably a mammal, and more preferably a human. In one embodiment, the present invention is directed to treatment or prevention of microbial infection of humans. In another embodiment, the present invention is directed to treatment or prevention of microbial infection of domestic animals, such as murine, rodent, feline or canine subjects, and farm animals, such as but not limited to bovine, equine and porcine subjects.

Specific indications or diseases that may be treated by the methods or compositions of the invention, include but are not limited to, acne, septicemia, toxic shock, gram-negative bacterial infections, endotoxin-related arthritis, gonorrhea, periodontal disease, spinal meningitis, infections of amniotic fluid, gram-positive bacterial infections, yeast infections, and fungal infections.

The present invention provides pharmaceutical compositions comprising ENP or ENP-derivatives. Such compositions may be administered either alone or in combination with other known drugs in vivo in a pharmaceutically or veterinarily acceptable carrier. If necessary, an adjuvant to facilitate absorption may be included in the formulation.

The term “carrier” as used herein means a synthetic or natural, inorganic or organic substance which is added to the ENP or ENP-derivatives to assist the active ingredients in reaching the location to be treated therewith or to facilitate storage, transportation and handling of the active ingredients.

Suitable liquid carriers may include, but not limited to, aromatic hydrocarbons such as benzene, toluene, and xylene; paraffinic hydrocarbons such as mineral oil and the like; halogenated hydrocarbons such as carbon tetrachloride, chloroform, dichloroethane and the like; ketones such as acetone, methyl ethyl ketone, etc.; ethers such as dioxane, tetrahydrofuran and the like; alcohols such as methanol, propanol, ethylene glycol and the like; or dimethyl formamide, dimethylsulfoxide, water, etc. Mixtures of any number of liquid carriers are also envisioned. Dissolution of lyophilized ENP or ENP-derivatives in un-buffered pyrogen-free distilled water or saline or phosphate buffered saline, may be achieved by adjusting the pH until the solution becomes water clear. For this reason, the preferred liquid carrier is pyrogen-free distilled water or saline adjusted to the appropriate pH to facilitate solubility of ENP or ENP-derivatives.

In order to enhance the effectiveness of the compound according to this invention, it is possible to use such adjuvants as given below, either singly or in combination, in accordance with the purpose of each application thereof while taking into consideration the form of their preparation and their field of application.

Exemplary adjuvants may include anionic surfactants such as alkyl sulfates, aryl sulfonates, succinates, polyethylene glycol, alkyl ether sulfates, and the like; cationic surfactants such as alkylamines, polyoxyethylene alkylamines, etc.; non-ionic surfactants such as polyoxyethylene glycol ethers, polyoxyethylene glycol esters, polyol esters and the like; and amphoteric surfactants. Encapsulation or microencapsulation of the active ingredient in liposome vesicles is also within the scope of this invention.

Examples of stabilizers, thickeners, lubricants and the like are isopropyl hydrogen-phosphate, calcium stearate, wax, casein, sodium alginate, serum albumin, other blood proteins, methylcellulose, carboxymethylcellulose, gum arabic, etc. It should be kept in mind that these ingredients are not limited to the recited examples.

Solutions or suspensions containing ENP or ENP-derivatives may also include the following components: a sterile diluent such as water for injection, saline solution, oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation may be enclosed in ampules, disposable syringes or multiple base vials made of glass or plastic.

Compositions of the invention can be administered orally. For such administrations, the pharmaceutical composition may be in liquid form, for example, solutions, syrups or suspensions, or may be presented as a drug product for reconstitution with water or other suitable vehicle before use. Such liquid preparations may be prepared by conventional means with pharmaceutically acceptable additives such as suspending agents (e.g., sorbitol syrup, cellulose derivatives or hydrogenated edible fats or oils); emulsifying agents (e.g., lecithin or acacia); non-aqueous vehicles (e.g., almond oil, oily esters, or fractionated vegetable oils); and preservatives (e.g., methyl or propyl-p-hydroxybenzoates or sorbic acid). The pharmaceutical compositions may take the form of, for example, tablets, capsules or pellets prepared by conventional means with pharmaceutically acceptable excipients such as binding agents (e.g., pregelatinized maize starch, polyvinyl pyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wefting agents (e.g., sodium lauryl sulphate). The tablets may be coated by methods well-known in the art.

For buccal administration, the compositions may take the form of tablets, troche or lozenge formulated in conventional manner.

Compositions, e.g., for oral or buccal administration, may be suitably formulated to give controlled release of the active compound. Such formulations may include one or more sustained-release agents known in the art, such as glyceryl mono-stearate, glyceryl distearate and wax.

Compositions of the invention also can be administered nasally or by inhalation. For nasal or inhalation administration, the compositions are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

Compositions of the invention may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt. Liposomes and emulsions are well known examples of delivery vehicles or carriers for hydrophilic drugs.

In addition to systemic administration, it is also within the scope of this invention to formulate ENP or ENP-derivatives into pharmaceutical compositions suitable for topical use to promote wound healing or to treat vaginal yeast infections. Topically applied ENP or ENP-derivatives, either alone or in combination with other antimicrobial agents, prevent or control gram-negative and gram-positive bacterial infections, yeast infections and growth of fungi.

A preferred embodiment includes topical formulations of ENP or ENP-derivatives alone, or in combination with known antibiotics, suitable for application to incisions or exposed tissue for the promotion of wound healing by curing or preventing bacterial, yeast or fungal infections. In another embodiment, ENP or ENP-derivatives are formulated into suppositories to treat vaginal yeast infections.

There are no limitations as to the type of wound or other trauma that can be treated, and these include: first, second and third degree burns, especially second and third degree; epidermal and internal surgical incisions, including those of cosmetic surgery; wounds, including lacerations, incisions, and penetrations; and epidermal ulcers including decubital (bed sores), diabetic, dental, hemophiliac, and varicose.

ENP or ENP-derivative compositions are applied to burns in the form of a sterile solution or lotion, preferably in combination with a physiological saline solution, or in the form of ointments or suspensions, preferably in combination with purified collagen. The compositions may also be impregnated into transdermal patches, plasters, bandages, or sterile implants preferably in a liquid or semi-liquid form.

Compositions for use in topical administration include, e.g., liquid or gel preparations suitable for penetration through the skin such as creams, liniments, lotions, ointments or pastes, and drops suitable for delivery to the eye, ear or nose.

According to the invention, creams, drops, liniments, lotions, ointments and pastes are liquid or semi-solid compositions for external application. Such compositions may be prepared by mixing the active ingredient(s) in powdered form, alone or in solution or suspension in an aqueous or non-aqueous fluid with a greasy or non-greasy base. The base may comprise complex hydrocarbons such as glycerol, various forms of paraffin, beeswax; a mucilage; a mineral or edible oil or fatty acids; or a macrogel. Such compositions may additionally comprise suitable surface active agents such as surfactants, and suspending agents such as agar, vegetable gums, cellulose derivatives, and other ingredients such as preservatives, antioxidants, etc.

Techniques and formulations for administering above-described compositions may be found in Remington's Pharmaceutical Sciences, Meade Publishing Col., Easton, Pa., latest edition.

A therapeutically effective dose of ENP or ENP-derivative is a dose that inhibits the growth of bacteria, yeast, or fungi when applied topically. The range of acceptable doses of ENP or ENP-derivative for topical application includes between about 0.01 and 10 weight percent. Where known antibiotics are combined with ENP or ENP-derivatives, their concentration can be varied up or down based on the range of known clinically acceptable concentrations for these drugs.

Initial dosing of ENP or ENP-derivatives either alone, or in combination with other agents, should be delivered topically to the therapeutic site at a concentration of about 0.5 weight percent. This dose can be thereafter adjusted up or down in line with clinical experience. Continued application or periodic reapplication of the compositions is indicated. The clinician will be expected to modify the dosage in accordance with clinical experience.

In a preferred embodiment, ENP or ENP-derivatives are topically applied at a concentration of between about 0.01 and 10 weight percent in a pharmaceutically acceptable carrier. Other preferred embodiments include application at concentrations of between about 0.1 and 1; 1 and 2; 2 and 5; 5 and 7; and 7 and 10 weight percent; most preferred is between about 1 and 2 weight percent.

The invention can be better understood by referring to the following examples, which are provided merely by way of exemplification and are not intended to limit the invention.

### 6. EXAMPLES

6.1 ENP Production In Yeast Expression Systems

ENP has been expressed in several yeast expression systems. ENP expression was achieved in these systems by transforming or integrating into the host cells an expression construct comprising a yeast promoter operably linked to a sequence encoding a fusion protein consisting of a yeast alpha-mating factor pre-pro-leader peptide fused to the N-terminal of a Limulus ENP. Two fusion protein coding sequences were constructed. One of these encodes fusion protein I and has the nucleotide and deduced amino acid sequences shown in FIG. 11 (SEQ ID NOs 1 and 2, respectively). As shown in FIG. 11, the leader peptide is from amino acid residues 1 to 4, and the Limulus ENP sequence is from amino acid residues 5 to 105.

The other fusion protein, fusion protein 11, comprises a yeast alpha-mating factor pre-pro leader peptide N-AsP-Gly-lle-Trp-Thr fused to the N-terminal of the Limulus ENP, whose amino acid sequence is shown in FIG. 13. Fusion protein 11 contains a Kex-2-like protease cleavage site. The expression construct encoding fusion protein 11 comprised SEQ. ID. NO:4.

Nucleotide sequences encoding the aforementioned two fusion proteins were designed to encode a known Limulus ENP amino acid sequence (i.e., SEQ ID NO:5) and used the codon preference of S. cerevisiae.

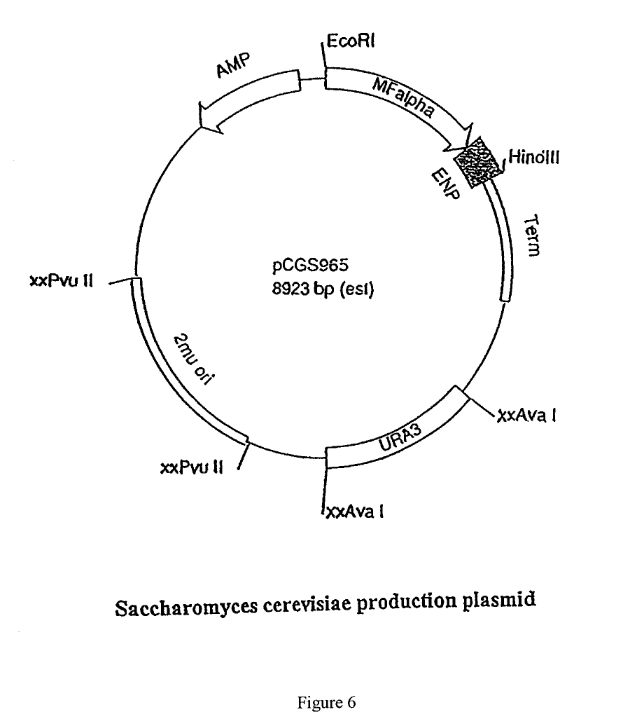

In one yeast expression system, an expression construct encoding the aforementioned fusion protein I was inserted into an autonomous replicating plasmid yielding pCGS965 (FIG. 6). This plasmid, in turn, was used to transform S. cerevisiae strain 2470. pCGS965 carries the gene for uracil production, which complements the host strain's auxotrophy for this base. Recombinant Limulus ENP has been expressed, purified, and characterized from pCGS965-transformed cells grown at the 500 liter scale. Fusion protein I was expressed and correctly processed, and the Limulus ENP secreted into the medium. The recombinantly produced Limulus ENP has biological activity identical to that of ENP isolated from Limulus amebocytes.

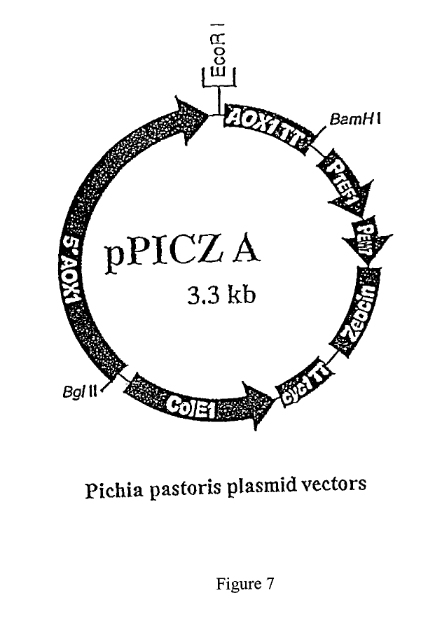

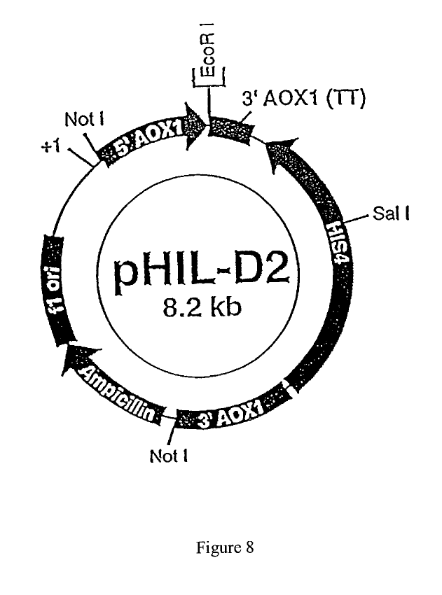

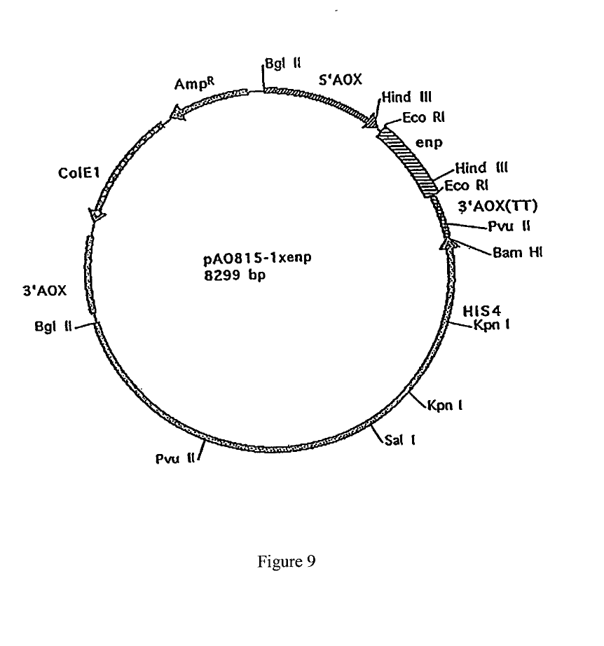

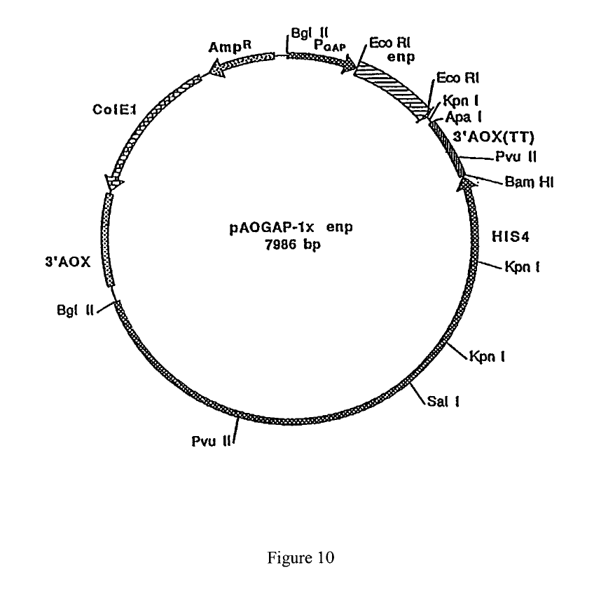

In another yeast expression system, expression constructs encoding fusion protein 1 or 11 were integrated into the genome of methylotrophic yeast Pichia pastoris. Plasmid vector pPIC9K containing an expression construct comprising the fusion protein 11 coding sequence (i.e., SEQ ID NO:4) and an AOX1 terminator sequence was constructed. Plasmid vectors pPICZ (FIG. 7), pHIL-D2 (FIG. 8), pAO815 (FIG. 9), and a novel hybrid plasmid, pGAPAO (FIG. 10), all containing expression constructs comprising a fusion protein I coding sequence (i.e., SEQ ID NO:1) and an AOX1 terminator sequence were constructed pPICZ, pHIL-D2, and pAO815 all use the tightly controlled, methanol-regulated AOX1 promotor to drive inducible expression of the fusion protein. pGAPAO is a novel expression vector which effects constitutive production of Limulus ENP in host cells grown under conditions that provide glucose instead of methanol as carbon source. The pGAPAO system utilizes glyceraldehyde-3-phosphate dehydrogenase promoter to drive the constitutive expression of the recombinant protein when glucose is provided as the carbon source.

Pichia host strain GS115^{his-}, SMD 1168, and X-33^{his+}, were transformed with the aforementioned plasmids. The transformations produced Pichia cells having the expression constructs integrated into the host chromosomal.

Analysis of the transformants showed that in some instances multiple copies of the expression constructs were inserted into the host genome without any apparently deleterious effect upon the host cell. Strains containing up to 6 copies of the expression construct encoding fusion protein 1, as verified by Southern blot analysis, were isolated and characterized. Methanol was used to induce expression of the Limulus ENP expression constructs driven by the AOX1 promoter (Scorer, 1993, Gene, 136:11-119: C. Scorer, 1994, Bio/technology 12:181-184). In each instance, the Pichia transformants produced biologically active Limulus ENP (e.g., having endotoxin binding and neutralizing activity).

Table 1 shows the levels of Limulus ENP found in the conditioned media of S. cerevisiae and various Pichia strains containing expression constructs encoding fusion protein 1. Pichia strains containing multiple integrated copies of the expression construct in some instances produced Limulus ENP at levels greater than 400 mg/liter.

TABLE 1
Specific Productivity of Recombinant Limulus ENP (rLENP) in Yeast

| | | | | LENP gene |
|---|---|---|---|---|
| | rLENP | Dry Cell Wt. | rLENP (mg/g | copy |
| Host strain | (mg/l) | (g/l) | dry cell weight) | #/genome |
| S. cerevisiae | 10 | 40 | 0.25 | N.A. |
| P. pastoris | | | | |
| Mut + 4 | 252 | 135 | 1.87 | 1 |
| GS 115-1 | 390 | 88 | 4.4 | 4 |
| SMD1168-1 | 445 | 93 | 4.78 | 6 |

Pichia containing Limulus ENP expression constructs may be grown at an acidic pH in an entirely synthetic media containing no protein hydrolysate supplements. The avoidance of amino acid supplements reduced both cost and more importantly, the endotoxin burden associated with these crude protein digests. Because ENP binds LPS, any exogenous endotoxin burden introduced into the fermentation or purification process can reduce the bioactivity of the secreted recombinant ENP. It is therefore critical to minimize the introduction of LPS into the process. Growing the expression construct-containing yeast cells in minimal media at acidic pH conditions, greatly reduced the chances for bacterial contamination and ENP product loss. These fermentation conditions also facilitated the direct, continuous application of clarified fermentation broth onto chromatography columns for purifying ENP or ENP-derivatives.

6.2. Isolation and Purification of Recombinantly Produced ENP

In the above-described expression systems, the Limulus ENP was secreted into the media via the alpha-mating factor pathway. Purification of the secreted Limulus ENP proceeded as follows. The initial purification step consisted of cell removal and clarification of the conditioned broth. This was accomplished by centrifugation, micro-filtration, or dynamic membrane filtration. Standard centrifugation equipment for either batch or continuous modes of operation were evaluated and found to be suitable for isolating secreted Limulus ENP from cells and cell debris. Adding NaCl to the fermentation broth to a concentration of about 0.2 M at harvest increased the amount of Limulus ENP recovered. It is believed that increasing the salt concentration of the harvest broth helped dissociate Limulus ENP from negatively-charged molecules present in the conditioned broth.

Standard micro-filtration techniques and equipment have been evaluated also for clarifying the conditioned fermentation broth. Tangential flow micro-filtration membranes consisting of 0.1-0.45 micron porosity were used successfully to remove cells while allowing Limulus ENP to pass through with the membrane filtrate. Membranes made of regenerated and derivatized nitrocellulose, polyether sulfone, nylon, etc, all effectively clarified high solid loads of up to 50% wet cell weight. Hollow fiber filters of 0.1 or 0.45 micron cut-off were also used successfully in the clarification process. The 0.45 micron hollow fiber diafiltration was carried out at high flow rate (1-5 liter/min) and low back pressure (5-10 psi).

Dynamic membrane filtration incorporating a magnetically coupled rotating nylon microfiltration membrane were incorporated into a continuous fermentation cycle where fermentation broth containing secreted Limulus ENP was removed from the fermentor at the same rate that nutrients were fed into the vessel. This process has been described for the production of recombinant lysozyme (Digan et al., Bio/Technology Vol. 7, 160-164, 1989). In this manner, the recombinant Limulus ENP were removed continuously from the fermentor vessel during operation as a chemostat. This process has the inherent advantage of reducing the exposure time of the secreted Limulus ENP to damaging proteases present in the conditioned broth.

After the clarification step, cells were discarded and the clarified broth was applied directly onto a capture chromatography column which binds Limulus ENP. In this manner, cell density in the fermentor was kept relatively low (i.e., between 150-350 g/l), and the culture was kept at mid-log growth phase and thus in a state of high productivity.

Ultrafiltration membranes also were used to size-fractionate, concentrate, and diafilter the clarified broth. Again, cellulosic and polymeric membranes in spiral wound, tangential sheets, and hollow-fiber configurations were evaluated and used as unit operations in downstream recovery steps. This purification step was achieved by collecting the filtrate from a 30,000 Dalton cut-off tangential flow ultrafiltration membrane cassette. The filtrate was concentrated by a 8,000 Dalton cut-off membrane, achieving a rapid size exclusion.

The cell free and/or ultrafiltered fermentation broth were then applied to a cation exchange column. Several support matrices and various chemistries have been evaluated including S, SP, and CM for this initial ion exchange step. Cross-linked agarose, ceramic, polymeric and silica based chromatography solid supports have been evaluated as capture columns for this process step. The preferred buffer system and pH for this capture step was 10 mM phosphate buffer at pH 3.0. The column was pre-equilibrated in 200 mM NaCl in phosphate buffer for application of the crude sample. This prevented binding by endogenous Pichia cationic proteins and served to increase the resin capacity specifically for Limulus ENP. The column was then washed with equilibration buffer and then step-eluted with 1 M and 2 M NaCl-phosphate buffer solutions. Limulus ENP was eluted in the 2 M salt fraction. This fraction was purified further on reverse phase resins such C4, C8, C18, and cyano-based columns; alternatively, hydrophobic interaction chromatography (HIC) based resins was employed. For reverse phase, the buffer systems consisted of pyrogen-free water plus 0.2% trifluoroacetic acid and mobile phase solvents such as iso-propanol, methanol, and acetonitrile also containing the ion coupling reagent TFA at 0.2%. A linear gradient from 0-100% organic phase was used to elute Limulus ENP from the solid support, or step elutions of organic solvent was employed to simplify the process. The Limulus ENP fraction typically eluted from a C4 bonded resin column in approximately 35% iso-propanol. Alternatively, the 2 M NaCl fraction from the cation exchange column was applied directly to a HIC column. Functional ligand chemistries capable of binding Limulus ENP in high salt without existence of ammonium sulfate were butyl and phenyl. Limulus ENP was captured in high salt and step eluted in PFW. The Limulus ENP-containing fraction was then ultrafiltered and concentrated and diafiltered into formulation buffer as previously described, or frozen and lyophilized directly. The lyophilized final Limulus ENP product was stored at −20° C.

6.3. Synergy of ENP and Antibiotics in Inhibiting Gram-Negative Bacteria

The growth inhibition experiments discussed below were carried out using the following procedure. An LAL 5000 spectrophotometer (Associates of Cape Cod) was used to monitor optical density changes over time when bacteria were grown in sterilized Luria broth (LB). The LAL 5000 can accommodate up to 32 individual 10×75 mm glass test tubes in its circular array. Automated temperature control was maintained at 37° C. for these experiments. LB was placed into sterile, oven depyrogenated glass test tubes. Tubes were then inoculated with cells from a log phase E. coli strain 25303 culture. Other gram-negative (E. coli strains Rosenbergii sp., K12, X10, HB101, Shewanella sp., Bortadella sp.) and gram-positive bacteria (e.g., S. aureus, and Bacillus spp.) were grown in this manner and changes in optical density were monitored over time. The optical density data was collected, stored and analyzed by computer which was interfaced with the LAL 5000.

6.3.1. ENP and Polymixin B Synergy

6.3.1.1. Experiment 1

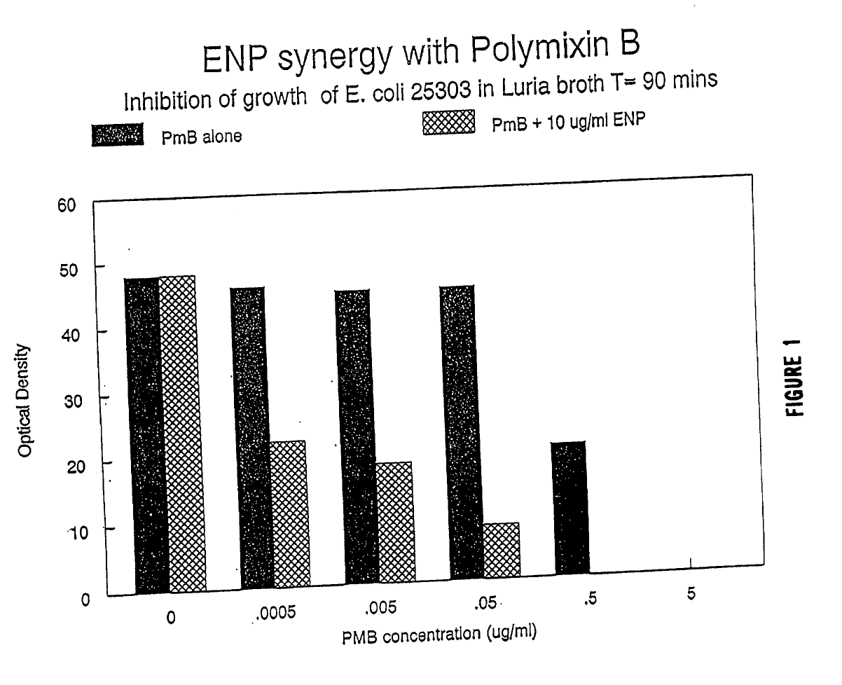

FIG. 1 shows the synergistic activity of Limulus ENP when used in conjunction with polymixin B (PMB) on inhibiting E. coli. PMB was serially diluted in ten fold increments in LB from 5 μg/ml to 0.0005 μg/ml. The tubes were then inoculated with cells of a log phase E. coli culture. Optical density of each tube was monitored via method described above. As the antibiotic concentration was diluted in LB, a corresponding increase in cell growth and optical density was observed. The lowest effective concentration of PMB alone which totally inhibited the growth of E. coli was 5 μg/ml When Limulus ENP was added at 10 μg/ml to the same concentration of PMB in LB, a potentiation of the inhibitory effect was observed. FIG. 1 demonstrates that treatment with 0.0005 μg/ml PMB with 10 μg/ml Limulus ENP inhibited E. coli growth (approximately 60%) to a level equivalent to that achieved by treatment with 0.5 μg/ml PMB alone. This represents a three log reduction in PMB concentration required to achieve the same inhibition of E. coli.

6.3.2. ENP and Gentamycin Synergy

6.3.2.1. Inhibition of E. coli

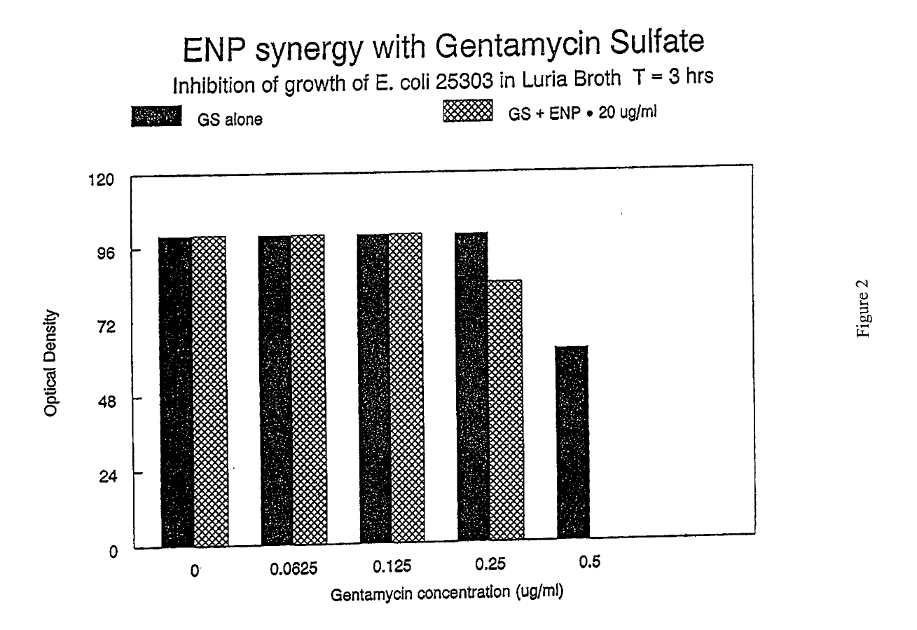

FIG. 2 shows the synergistic activity of Limulus ENP when used in conjunction with gentamycin sulfate (GMS) on inhibiting E. coli. GMS was serially diluted in two fold increments in LB from 1.0 μg/ml to 0.0625 μg/ml. The tubes were then inoculated with cells of a log phase E. coli culture. Optical density of each tube was monitored via method described above. As the antibiotic concentration was decreased, a corresponding increase in cell growth and optical density was observed. The effective concentration of GMS alone which substantially inhibits the growth of E. coli was 0.5 μg/ml. When Limulus ENP was added at 20 μg/ml to the antibiotic-containing cultures, a potentiation of GMS inhibitory activity was observed at GMS concentration of 0.25 μg/ml.

6.3.2.2. Inhibition of Bortadella sp.

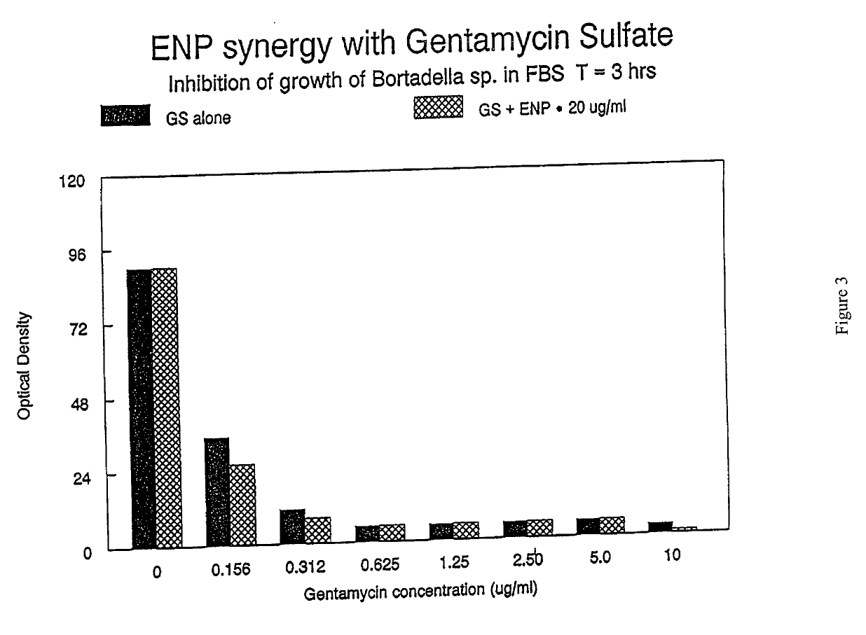

FIG. 3 shows the synergistic activity of Limulus ENP when used in conjunction with GMS on inhibiting Bortadella sp. GMS was serially diluted in two fold increments in LB from 10 μg/ml to 0.156 μg/ml. The tubes were then inoculated with cells from a log phase culture of Bortadella sp. Optical density of each tube was monitored via method described above. As the antibiotic concentration was decreased, a corresponding increase in cell growth and optical density was observed. The lowest effective concentration of GMS alone which almost totally inhibited the growth of Bortadella sp. was 10 μg/ml. When Limulus ENP is added at 20 μg/ml, a potentiation of the inhibitory effect was observed at 0.156 μg/ml and 0.312 μg/ml of GMS.

6.3.3. ENP and Tetracycline Synergy

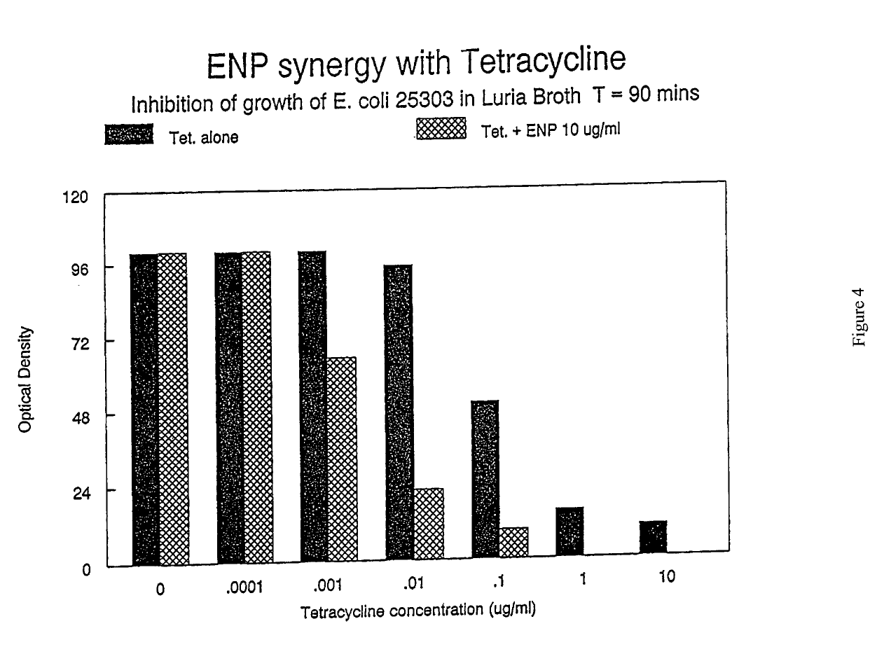

FIG. 4 shows the synergistic activity of Limulus ENP when used in conjunction with tetracycline (TET) on inhibiting E. coli. TET was serially diluted with LB in ten-fold increments from 10 μg/ml to 0.0001 μg/ml. The tubes were then inoculated with cells from a log phase E. coli culture. Optical density of each tube was monitored via method described above. As TET concentration was decreased, the cell growth increased. When Limulus ENP was added at 10 μg/ml to the TET containing cultures, a potentiation of the inhibitory activity was observed at TET concentrations of 0.001, 0.01 and 0.1 μg/ml. FIG. 4 demonstrates that 0.1 μg/ml TET plus 10 μg/ml Limulus ENP inhibited E. coli to nearly the same degree as when TET was used alone at 1 to 10 μg/ml. This represents a two to three orders of magnitude enhancement of the inhibitory activity of TET.

6.4. Synergy of ENP And Antibiotics In Inhibiting Gram-Positive Bacteria

ENP also synergistically enhances the inhibitory activity of antibiotics against gram-positive bacteria such as S. aureus and Bacillus spp. The mechanism of this synergistic action is not known, as the cell walls of gram-positive bacteria do not contain the lipid A moiety which ENP has been shown to bind. It is hypothesized that ENP disrupts the cell membrane. The mode of action of ENP may be similar to that of CAP-18 and BPI, other cationic and LPS binding, anti-microbial proteins. Peptide fragments of these proteins have also been shown to neutralize endotoxins in vitro (Ogata et al, Infection and Immunity, June 1997, p. 2160-2167). Such permeablization of the cell membrane may account for the synergistic effect of ENP through enhancing the entry of antibiotics or weakening of the gram-positive bacteria.

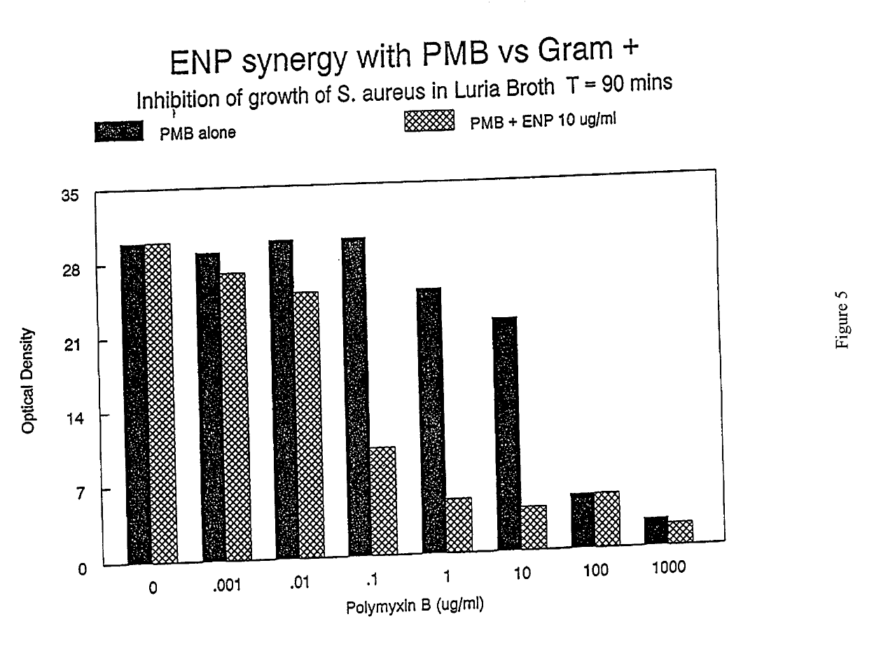

FIG. 5 shows the synergistic activity of Limulus ENP when used in conjunction with PMB on inhibiting S. aureus. PMB was serially diluted in ten fold dilutions in LB. The dilution tubes were then inoculated with cells from a log phase culture of S. aureus. An identical second series of dilution tubes of PMB was prepared in the same manner with an addition of Limulus ENP included at a final concentration of 10 ug/ml. Optical density of each tube is monitored via method described below. FIG. 5 shows the optical density values for the two sets of dilution tubes at 90 minutes after inoculation. The results show that as the PMB concentration was reduced, the optical density of the cell cultures increased. Further, when Limulus ENP at 10 ug/ml is included with PMB, there is a subsequent reduction in growth at all concentrations of PMB. Significantly, PMB at 1 μg/ml plus LALF at 10 μg/ml, has approximately the same inhibitory activity as that of PMB alone at 100 μg/ml. This represents a two orders of magnitude enhancement of PMB's inhibitory activity against this bacteria due to the synergistic effect of Limulus ENP.

6.5. ENP Suppresses the Release of Endotoxin from Inhibited Bacteria

Antibiotic-inhibited gram-negative bacteria release endotoxins into the medium. ENP suppresses this release. This was demonstrated in E. coli cultures that were inhibited by PMB or GMS. E. coli cultures treated with antibiotic alone or in combination with Limulus ENP were prepared and incubated as described in Sections 6.3.1 and 6.3.2, above. The endotoxin levels present in the cultures were measured using the standard Limulus amebocyte lysate (LAL) assay. Dramatic differences in measured levels of LPS were observed between cultures treated with PMB alone and those treated with PMB plus Limulus ENP. Endotoxin levels were greatly reduced and much lower in tubes where PMB was combined with Limulus ENP than tubes treated with PMB alone. The results are shown in Table 2.

Similar effects were observed with cultures treated with GMS with and without Limulus ENP. Those results are shown in Table 3.

TABLE 2
ENP Suppression of Endotoxin Levels in Cultures Inhibited by PMB

| SAMPLE | ENDOTOXIN LEVEL |
|---|---|
| PMB at 5 pg/ml | 3.8 μg/ml |
| PMB at 5 μg/ml, with Limulus ENP | 0.0318 μg/ml |

TABLE 3
Limulus ENP Suppression of Endotoxin Levels in Cultures Inhibited
by GMS

| SAMPLE | ENDOTOXIN LEVEL |
|---|---|
| GMS at 1 μg/ml | 132 μg/ml |
| GMS at 1 μg/ml, with Limulus ENP | 0.0011 μg/ml |

The detectable endotoxin levels in cultures treated with Limulus ENP plus antibiotic compositions were orders of magnitude lower than those of cultures treated with antibiotics only compositions. Similar experiments also have been carried out with serum-containing media. The presence of serum proteins did not adversely affect the biological activity, the antimicrobial properties or the endotoxin binding activity of Limulus ENP. Specifically, serum proteins did not altered Limulus ENP ability to potentiate the inhibitory activity of antibiotics or to lower the levels of endotoxins released from inhibited bacteria.

Various publications are cited herein, the disclosures of which are incorporated by reference in their entireties.

What is claimed is:

1. An pharmaceutical composition comprising (a) one or several endotoxin inactivating proteins, and (b) one or several antibiotics; wherein the endotoxin inactivating protein is an endotoxin neutralizing protein (ENP) of a horseshoe crab or a derivative of the ENP, which derivative comprises an endotoxin binding and neutralizing domain of the ENP.

2. The pharmaceutical composition of claim 1, wherein the horseshoe crab is Limulus polyphemus, Tachypleus gigas, Tachypleus tridentatus or Carcinoscropius rotundicauda.

3. The pharmaceutical composition of claim 2, wherein the horseshoe crab is Limulus polyphemus.

4. The pharmaceutical composition of claim 3, wherein the endotoxin inactivating protein comprises the sequence of SEQ ID NO:3.

5. The pharmaceutical composition of claim 4, wherein the endotoxin inactivating protein comprises the sequence of SEQ ID NO:5.

6. The pharmaceutical composition of claim 4, wherein the endotoxin inactivating protein comprises the sequence of SEQ ID NO:2.

7. The pharmaceutical composition of claim 1, wherein the antibiotic inhibits a bacterial species.

8. The pharmaceutical composition of claim 7, wherein the antibiotic inhibits a gram-negative bacterial species.

9. The pharmaceutical composition of claim 7, wherein the antibiotic inhibits a gram-positive bacterial species.

10. The pharmaceutical composition of claim 7, wherein the antibiotic is selected from the group consisting of polymyxin B, ampicillin, amoxicillin, penicillin G, penicillin A, penicillin V, tetracycline, erythromycin, spectinomycin, cefoxitin, trimethoprimsulfamethoxazole, chloramphenicol, rifampin, minocycline, sulfonamides, nitrofurantoin, gentamicin, cefamandole, carbenicillin, ticarcillin, tobramycin, amikacin, cephalosporin, cefoxitin, streptomycin, clindamycin, vancomycin cloxacillin, dicloxacillin, methicillin, nafcillin, oxacillin, piperacillin, meziocillin, cephalexin, cephradine, cefaclor, cefadroxil, cefixime, cefuroxime, cefprozil, cephalothin, cefrazolin, cephapirin, cefoperazone, ceftrixone, tetracycline, chlortetracycline, oxytetracyclione, demeclocycline, minocycline, methacycline, lincomycin, clarithromycin, azithromycin, metronidazole, and metronidazole.

11. The pharmaceutical composition of claim 8, or 9, which further comprises a pharmaceutically acceptable carrier.

12. A pharmaceutical composition comprising (a) one or several endotoxin inactivating proteins, (b) one or several antibiotics, and (c) a pharmaceutically acceptable carrier; wherein the endotoxin inactivating protein is an endotoxin neutralizing protein (ENP) of a horseshoe crab or a derivative of the ENP, which derivative comprises the endotoxin binding and neutralizing domain of the ENP.

13. The pharmaceutical composition of claim 12, wherein the antibiotic is effective against gram-negative bacterial infections.

14. The pharmaceutical composition of claim 12, wherein the antibiotic is effective against gram-positive bacterial infections.

15. A method for treating or preventing a microbial infection, comprising administering to a patient an effective amount of one or several endotoxin inactivating proteins, and an effective amount of one or several antibiotics; wherein the endotoxin inactivating protein is an endotoxin neutralizing protein (ENP) of a horseshoe crab or a derivative of the ENP, which derivative comprises the endotoxin binding and neutralizing domain of the ENP.

16. The method according to claim 15, wherein the horseshoe crab is Limulus polyphemus, Tachypleus gigas, Tachypleus tridentatus or Carcinoscropius rotundicauda.

17. The method according to claim 16, wherein the horseshoe crab is Limulus polyphemus.

18. The method according to claim 17, wherein the endotoxin inactivating protein comprises the sequence of SEQ ID NO:3.

19. The method according to claim 18, wherein the endotoxin inactivating protein comprises the sequence of SEQ ID NO:5.

20. The method according to claim 19, wherein the endotoxin inactivating protein comprises the sequence of SEQ ID NO:2.

21. The method according to claim 15, wherein the microbial infection is a bacterial infection and the method comprises administering an antibiotic that is effective against the bacterial infection.

22. The method according to claim 21, wherein the microbial infection is a gram-negative bacterial infection and the method comprises administering an antibiotic that is effective against the gram-negative bacterial infection.

23. The method according to claim 21, wherein the microbial infection is a gram-positive bacterial infection and the method comprises administering an antibiotic that is effective against the gram-positive bacterial infection.

24. The method according to claim 21, wherein the antibiotic is selected from the group consisting of polymyxin B, ampicillin, amoxicillin, penicillin G, penicillin A, penicillin V, tetracycline, erythromycin, spectinomycin, cefoxitin, trimethoprimsulfamethoxazole, chloramphenicol, rifampin, minocycline, sulfonamides, nitrofurantoin, gentamicin, cefamandole, carbenicillin, ticarcillin, tobramycin, amikacin, cephalosporin, cefoxitin, streptomycin, clindamycin, vancomycin cloxacillin, dicloxacillin, methicillin, nafcillin, oxacillin, piperacillin, meziocillin, cephalexin, cephradine, cefaclor, cefadroxil, cefixime, cefuroxime, cefprozil, cephalothin, cefrazolin, cephapirin, cefoperazone, ceftrixone, tetracycline, chlortetracycline, oxytetracyclione, demeclocycline, minocycline, methacycline, lincomycin, clarithromycin, azithromycin, metronidazole, and metronidazole.

25. The method according to claim 15, wherein the patient is a warm-blooded animal.

26. The method according to claim 25, wherein the patient is a human.

27. A method for treating a gram-negative bacterial infection, comprising administering to a patient an effective amount of one or several endotoxin inactivating proteins, and an effective amount of one or several antibiotics that are effective against the gram-negative bacterial infection; wherein the endotoxin inactivating protein is an endotoxin neutralizing protein (ENP) of a horseshoe crab or a derivative of the ENP, which derivative comprises the endotoxin binding and neutralizing domain of the ENP.

28. A method for treating a gram-positive bacterial infection, comprising administering to a patient an effective amount of one or several endotoxin inactivating proteins, and an effective amount of one or several antibiotics that are effective against the gram-positive bacterial infection; wherein the endotoxin inactivating protein is an endotoxin neutralizing protein (ENP) of a horseshoe crab or a derivative of the ENP, which derivative comprises the endotoxin binding and neutralizing domain of the ENP.
